(19) United States
(12) Patent Application Publication
(10) Pub. No.: US 2026/0133548 A1
(43) Pub. Date: May 14, 2026

(54) SYSTEMS AND METHODS FOR MATERIAL SUBGRAPH MODEL PRE-TRAINING THROUGH INDEPENDENT AND INTEGRATED FORCE FIELD

(71) Applicant: Samsung Display Co., Ltd., Yongin-si (KR)

(72) Inventors: Shuhui Qu, Fremont, CA (US); Cheol Woo Park, San Jose, CA (US); Qisen Cheng, Sunnyvale, CA (US); Janghwan Lee, Pleasanton, CA (US)

(21) Appl. No.: 19/171,042

(22) Filed: Apr. 4, 2025

Related U.S. Application Data
(60) Provisional application No. 63/719,438, filed on Nov. 12, 2024.

(51) Int. Cl. G05B 13/04; G05B 13/02; G16C 20/50; G16C 20/70; G16C 60/00

(52) U.S. Cl. CPC G05B 13/048; G05B 13/027; G16C 20/50; G16C 20/70; G16C 60/00

(57) ABSTRACT

A system and a method are disclosed for force field prediction using subgraph modeling. A method utilizes artificial intelligence (AI)-based force field subgraph models to incorporate robust force field predictions relating to molecules of a material to improve the accuracy of downstream tasks (e.g., material property predictions) related to the production of a display related product. Force field predictions improve the performance of display related products by considering the impacts of force fields and/or utilizing materials deemed suitable. Methods include generating subgraphs, where each of the subgraphs includes a molecular substructure of a material, applying an AI-based model to the subgraphs to generate at least one force field prediction based on the molecular substructure of the material, and applying the AI-based model to a graph of a structure of a molecule of the material to generate a force field prediction based on the structure of the molecule of the material.

### CROSS-REFERENCE TO RELATED APPLICATION

This application claims the priority benefit under 35 U.S.C. § 119(e) of U.S. Provisional Application No. 63/719,438, filed on Nov. 12, 2024, the disclosure of which is incorporated by reference in its entirety as if fully set forth herein.

### BACKGROUND

### 1. Field

Aspects of some embodiments of the present disclosure generally relate to machine learning and/or artificial intelligence. More particularly, the subject matter disclosed herein relates to determining force field data for display related products based on artificial intelligence.

### 2. Description of the Related Art

Material property prediction may involve estimating the physical, chemical, mechanical, and/or optical properties of materials that can be used in display related products. A force field of a molecule may be correlated with other properties of the material, a thus may cause analyzing and/or determining the force field related to a molecular structure to become a valuable operation for downstream material property prediction tasks. Therefore, it may be desirable to utilize computational models, for example artificial intelligence-based models, to generate force field predictions that may in turn improve the accuracy and robustness of material property predictions, and can be utilized in various aspects of the design and/or manufacture of display related products, including material selection, performance optimization, and innovation in display technology.

The above information disclosed in this Background section is for enhancement of understanding of the background of the present disclosure, and therefore, it may contain information that does not constitute prior art.

### SUMMARY

Aspects of some embodiments of the present disclosure generally relate to material property prediction and/or analysis. For example, aspects of some embodiments of the present disclosure generally relate to improvements to the accuracy and efficiency of material property predictions by utilizing artificial intelligence and/or material subgraph models.

Material property prediction may involve estimating the physical, chemical, mechanical, and/or optical properties of materials that can be used in display related products. Therefore, it may be desirable to utilize computational models, for example artificial intelligence-based models, to generate material property predictions that can then be utilized in various aspects of the design and/or manufacture of display related products, including material selection, performance optimization, and innovation in display technology. However, there may be issues associated with applying artificial intelligence (AI) techniques to material property prediction, in a manner that maintains the robustness and/or efficiency of AI and can scale to the relatively large size and complexity of the molecular structure of materials that may be used for display related products.

Aspects of some embodiments of the present disclosure relate to systems and methods for AI-based material subgraph models, including the decomposition of the molecular structure of materials, generating graphs of subgraphs, and implementing subgraph modeling in a manner that may scale AI-based models to large and/or complex molecules and enhances their expressive power. Thus, the disclosed embodiments may improve the overall performance of display related products by utilizing materials deemed most suitable and/or efficient.

In some embodiments, a method includes generating by a processor, subgraphs, each of the subgraphs including a molecular substructure of a material; applying, by the processor, an AI-based model to the subgraphs to generate at least one force field prediction based on the molecular substructure of the material; applying, by the processor, the AI-based model to a graph of a structure of a molecule of the material to generate a force field prediction based on the structure of the molecule of the material; determining, by the processor, a function related to the material for production of a device based on the at least one force field prediction based on the molecular substructure of the material and the force field prediction based on the structure of the molecule of the material; and transmitting, by the processor, a signal to a component to control the component to execute the function related to the material for the production of the device.

In some embodiments, the method may further include applying, by the processor, the AI-based model to the subgraphs to generate a material property prediction, and generating the at least one force field prediction based on the molecular substructure of the material and generating the force field prediction based on the structure of the molecule of the material is a pre-training process of the AI-based model.

In some embodiments, the method may further include decomposing the graph of the structure of the molecule into the molecular substructures.

In some embodiments, the decomposing includes Breaking Retrosynthetically Interesting Chemical bonds (BRIC) decomposition.

In some embodiments, the method may further include applying a force field calculation function to the subgraphs and obtaining at least one substructure conformation.

In some embodiments, the method may further include generating subgraph embeddings by processing the subgraphs and the at least one substructure conformation by a three-dimensional (3D) graph neural network.

In some embodiments, the at least one force field prediction based on the molecular substructure of the material is generated based on the subgraph embeddings.

In some embodiments, the method may further include applying a force field calculation function to the graph of the structure of the molecule and obtaining a molecule conformation.

In some embodiments, the method may further include generating subgraph embeddings by processing the subgraphs and the molecule conformation by a 3D graph neural network.

In some embodiments, the method may further include generating a graph of subgraphs based on the subgraph embeddings.

In some embodiments, the graph of subgraphs may include nodes and edges.

In some embodiments, each of the nodes correspond to one of the subgraphs and a value for each of the nodes correspond to one of the embeddings of the subgraphs.

In some embodiments, each of the edges represent a relationship between the subgraphs.

In some embodiments, the method may further include generating updated subgraph embeddings based on the graph of subgraphs processing the graph of subgraphs by a graph neural network.

In some embodiments, the force field prediction based on the structure of the molecule of the material is generated based on the updated subgraph embeddings.

In some embodiments, the AI-based model analyzes the graph of subgraphs and models relationships between the subgraphs.

In some embodiments, the device comprises an organic light-emitting diode (OLED) display device.

In some embodiments, a device includes: one or more processors that are configured to perform: generating subgraphs, each of the subgraphs including a molecular substructure of a material; applying an AI-based model to the subgraphs to generate at least one force field prediction based on the molecular substructure of the material; applying the AI-based model to a graph of a structure of a molecule to generate a force field prediction based on the structure of the molecule of the material; determining a function related to the material for production of a device based on the at least one force field prediction based on the molecular substructure of the material and the force field prediction based on the structure of the molecule of the material; and transmitting a signal to a component to control the component to execute the function related to the material for the production of the device.

In some embodiments, the device is further configured to apply the AI-based model to the subgraphs to generate a material property prediction and generating the at least one force field prediction based on the molecular substructure of the material and generating the force field prediction based on the structure of the molecule of the material is a pre-training process of the AI-based model.

In some embodiments, a system includes: a processing circuit; and a non-volatile memory storing instructions, which, based on being executed by the processing circuit, cause the processing circuit to perform: generating subgraphs, each of the subgraphs including a molecular substructure of a material; applying an AI-based model to the subgraphs to generate at least one force field prediction based on the molecular substructure of the material; applying the AI-based model to a graph of a structure of a molecule of the material to generate a force field prediction based on the structure of the molecule of the material; determining a function related to the material for production of a device based on the at least one force field prediction based on the molecular substructure of the material and the force field prediction based on the structure of the molecule of the material; and transmitting a signal to a component to control the component to execute the function related to the material for the production of the device.

### BRIEF DESCRIPTION OF THE DRAWINGS

In the following section, the aspects of the subject matter disclosed herein will be described with reference to exemplary embodiments illustrated in the figures.

FIG. 1 is a block diagram depicting a system (e.g., a factory) for producing products (e.g., electronic devices, such as organic light-emitting diode (OLED) display devices) utilizing AI-based force field subgraph models, according to some embodiments of the present disclosure.

FIG. 2 is a block diagram depicting a computer device for force field prediction, including an example force field subgraph modeling circuit, according to some embodiments of the present disclosure.

FIG. 3 is a diagram depicting an example of independent force field aspects relating to a molecular structure for a material and integrated force field aspects relating to the molecular structure for the material, according to some embodiments of the present disclosure.

FIG. 4 is a diagram depicting an example process for decomposing and applying force field calculations implemented by the force field subgraph modeling circuit of FIG. 2, according to some embodiments of the present disclosure.

FIG. 5 is a diagram depicting an example process for generating subgraph embeddings and a graph of subgraphs implemented by the force field subgraph modeling circuit of FIG. 2, according to some embodiments of the present disclosure.

FIG. 6 is a diagram depicting an example process for generating the force field predictions implemented by the subgraph modeling circuit of FIG. 2, according to some embodiments of the present disclosure.

FIG. 7 is a flow chart depicting example operations of a method for producing a device (e.g., OLED display devices) utilizing AI-based force field subgraph models, according to some embodiments of the present disclosure.

FIG. 8 is a block diagram of an electronic device in a network environment 800, according to some embodiments of the present disclosure.

### DETAILED DESCRIPTION

In the following detailed description, numerous specific details are set forth in order to provide a thorough understanding of the disclosure. It will be understood, however, by those skilled in the art that the disclosed aspects may be practiced without these specific details. In other instances, well-known methods, procedures, components and circuits have not been described in detail to not obscure the subject matter disclosed herein.

Reference throughout this specification to “one embodiment” or “an embodiment” means that a particular feature, structure, or characteristic described in connection with the embodiment may be included in at least one embodiment disclosed herein. Thus, the appearances of the phrases “in one embodiment” or “in an embodiment” or “according to one embodiment” (or other phrases having similar import) in various places throughout this specification may not necessarily all be referring to the same embodiment. Furthermore, the particular features, structures or characteristics may be combined in any suitable manner in some embodiments (e.g., in one or more embodiments). In this regard, as used herein, the word “exemplary” means “serving as an example, instance, or illustration.” Any embodiment described herein as “exemplary” is not to be construed as necessarily preferred or advantageous over other embodiments. Additionally, the particular features, structures, or characteristics may be combined in any suitable manner in one or more embodiments. Also, depending on the context of discussion herein, a singular term may include the corresponding plural forms and a plural term may include the corresponding singular form. Similarly, a hyphenated term (e.g., “two-dimensional,” “pre-determined,” “pixel-specific,” etc.) may be occasionally interchangeably used with a corresponding non-hyphenated version (e.g., “two dimensional,” “predetermined,” “pixel specific,” etc.), and a capitalized entry (e.g., “Counter Clock,” “Row Select,” “PIXOUT,” etc.) may be interchangeably used with a corresponding non-capitalized version (e.g., “counter clock,” “row select,” “pixout,” etc.). Such occasional interchangeable uses shall not be considered inconsistent with each other.

Also, depending on the context of discussion herein, a singular term may include the corresponding plural forms and a plural term may include the corresponding singular form. It is further noted that various figures (including component diagrams) shown and discussed herein are for illustrative purpose only, and are not drawn to scale. For example, the dimensions of some of the elements may be exaggerated relative to other elements for clarity. Further, if considered appropriate, reference numerals have been repeated among the figures to indicate corresponding and/or analogous elements.

The terminology used herein is for the purpose of describing some example embodiments only and is not intended to be limiting of the claimed subject matter. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that when an element or layer is referred to as being on, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

The terms “first,” “second,” etc., as used herein, are used as labels for nouns that they precede, and do not imply any type of ordering (e.g., spatial, temporal, logical, etc.) unless explicitly defined as such. Furthermore, the same reference numerals may be used across two or more figures to refer to parts, components, blocks, circuits, units, or modules having the same or similar functionality. Such usage is, however, for simplicity of illustration and ease of discussion only; it does not imply that the construction or architectural details of such components or units are the same across all embodiments or such commonly-referenced parts/modules are the only way to implement some of the example embodiments disclosed herein.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the term “module” refers to any combination of software, firmware and/or hardware configured to provide the functionality described herein in connection with a module. For example, software may be embodied as a software package, code and/or instruction set or instructions, and the term “hardware,” as used in any implementation described herein, may include, for example, singly or in any combination, an assembly, hardwired circuitry, programmable circuitry, state machine circuitry, and/or firmware that stores instructions executed by programmable circuitry. The modules may, collectively or individually, be embodied as circuitry that forms part of a larger system, for example, but not limited to, an integrated circuit (IC), system on-a-chip (SoC), an assembly, and so forth.

In recent years, the display industry has been focused on developing cutting-edge, next-generation display materials for products. The aim may be to enable new types of high-efficiency and low-cost display related products. A critical process to realizing improved products, may be obtaining material properties efficiently and accurately. Efficient determination of the properties and/or characteristics of potential materials to be used for fabricating products may accelerate the development and optimization of new materials.

Density Functional Theory (DFT) simulations may be used to obtain the material properties based on physical constraints before any experimental validation. However, the accuracy and/or reliability of the predictions of material properties provided by DFT simulations (and other conventional mechanisms) may be limited. For example, DFT approaches may experience delays (relatively long time periods to reach steady-state solutions). The computational inefficiency of some current material property analysis mechanisms, such as DFT, may limit their application in the rapid discovery of new materials.

A force field of a molecule may be correlated with other properties of the material, a thus may cause analyzing and/or determining the force field related to a molecular structure to become a valuable operation for downstream material property prediction tasks. Compared to the DFT tools, force field calculation (and/or force field minimization) may require less computational resources, many may have a comparatively increased ease of use and less cost-effectiveness to obtain. Also, different force field minimization algorithms may yield varying results, offering diverse representations of molecular behavior that may require some robustness of the tools used to calculate and/or model the differing behaviors. While force field minimization for small molecules may be relatively rapid, it may be time-consuming for larger molecules due to their complexity, which may be the case for large molecules (e.g., approximately 100 or greater atoms) of materials used for producing display related products.

Additionally, there may be some AI-based approaches currently utilized to provide automated material property analysis and/or prediction tools. Nonetheless, these AI-based models may not effectively capture the various relationships, including spatial arrangements of atoms in the three-dimensional (3D) space, that may be associated with the complex molecular structure of materials, resulting in a loss of crucial relational information.

These AI-based models may not be suitable for capturing long-range interactions, which may be a limitation that can reduces the model's expressiveness, leading to lower performance when analyzing large molecules. Furthermore, the sampling approaches for these AI-based models may rely on conventional modeling functions (e.g., node and/or edge removal) which may not be suitable for leveraging chemical decomposition techniques, and thereby may reduce the model's ability to efficiently handle large molecules (e.g., molecules with hundreds of atoms), where many materials used for display related devices may include large molecules. For example, there are some existing technologies involving 3D Graph Neural Networks (GNNs) that utilize the 3D molecular conformations as input, which have demonstrated some improved performance in property prediction compared to conventional GNNs. Nonetheless, some current approaches that attempt to leverage 3D GNN capabilities may be sensitive to perturbations in the molecular conformation, which can lead to significant variations in the predicted properties.

To improve the force field analysis and/or predictions for materials that may be used to produce display related products, and in turn enhanced display related products, the embodiments implement functions related to generating, pre-training, training, and utilizing AI-based subgraph models that may scale to large and complex molecules and may be further used to improve accuracy and/or performance of material property predictions. As alluded to above, material utilized for producing display related products may often involve large and/or complex molecules, and thus may require AI-based models that can handle such complexity. The AI-based force field subgraph models, as disclosed herein, are implemented to scale to large molecules by leveraging subgraphs. For example, the AI-based subgraph models may leverage subgraph decomposition for material (e.g., chemical) molecular structure instead of relying on more conventional AI modeling techniques (e.g., edge deletion, node deletion, etc.).

The force field subgraph modeling functions, as disclosed herein, may leverage force field calculations in a manner that improves model performance, developing AI-based models that can capture independent force field aspects for molecules and integrated force field aspects for molecules, and thus may enable force field information to be optimally incorporated to improve the accuracy and robustness of property predictions for various molecular sizes. In addition, force field subgraph modeling, as disclosed herein, may strengthen the stability and reliability of 3D GNNs to model molecular structure, and in a manner that may ensure more consistent performance even with variations in molecular conformations. Thus, systems and/or functions that may incorporate (e.g., model pre-training) the enhanced forced field predictions generated by the disclosed force field subgraph modeling may also achieve higher performance in material property prediction.

Aspects of some embodiments of the present disclosure provide for AI-based force field subgraph models to mitigate (e.g., to overcome) the aforementioned issues by implementing functions that may include: subgraph selection, which may involve utilizing a domain-specific subgraph sampler to identify and/or select meaningful substructures within a molecular graph based on specialized chemical principles (e.g., Breaking Retrosynthetically Interesting Chemical bonds (BRIC), hierarchical decomposition strategies, etc.); subgraph representation, which may include representing each subgraph as a corresponding node within a constructed graph (referred to herein as a graph of subgraphs), where edges between nodes may denote interactions or relationships between the corresponding subgraphs; interaction modeling, which may involve capturing and/or modeling the interactions (and dependencies) between the subgraphs within the graph of subgraphs to preserve relational information and enable the representation of complex structural patterns; and force field property prediction, which may involve leveraging subgraphs for modeling 3D information related to molecules, and for analyzing independent force field aspects and/or integrated force field aspects that may be impacted by molecular structure to generate force field predictions with improved accuracy and robustness.

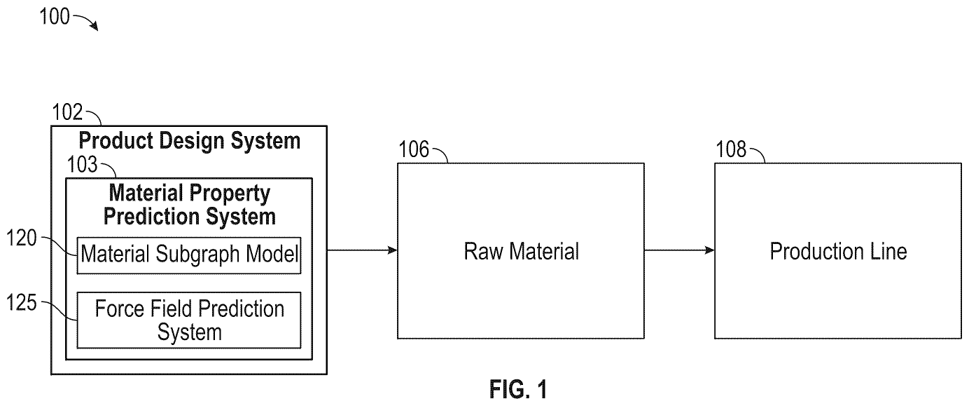

FIG. 1 is a block diagram depicting a system 100 (e.g., a factory) for producing display related products (e.g., electronic devices, such as organic light-emitting diode (OLED) display devices), according to some embodiments of the present disclosure.

Manufacturing products (e.g., in a factory or a production line) may include various processes to ensure certain quality standards are satisfied. In some embodiments, the system 100 (e.g., the factory) of FIG. 1 may produce products (e.g., electronic devices, such as display devices, integrated circuits, and/or the like). As seen in FIG. 1, the system 100 may include a production line 108. The production line 108 may include machines, machinery, and/or devices that take raw materials and/or components 106 as inputs and assembles, constructs, and/or produces one or more products, such as the display devices (e.g., OLED display devices).

In manufacturing some display related products (e.g., OLED display devices), material property predictions may be used to identify and select the most suitable organic materials for each layer of the device. The product design system 102 may include a material property prediction system 103, which may be implemented as a computer device having the capability to generate, train, and/or utilize a material subgraph model 120. The material subgraph model 120 may be an artificial intelligence (AI) and/or machine learning (ML) based model (also referred to herein as “neural networks”) that can be generated, trained, and/or utilized in accordance with the subgraph modeling functions, as disclosed herein, and may realize improved efficiency and/or accuracy in predicting material properties. The production design system 102 may then leverage the enhanced accuracy and/or prediction performance of the material property prediction system 103 for its functions relating to the design, testing, and/or production of display related products.

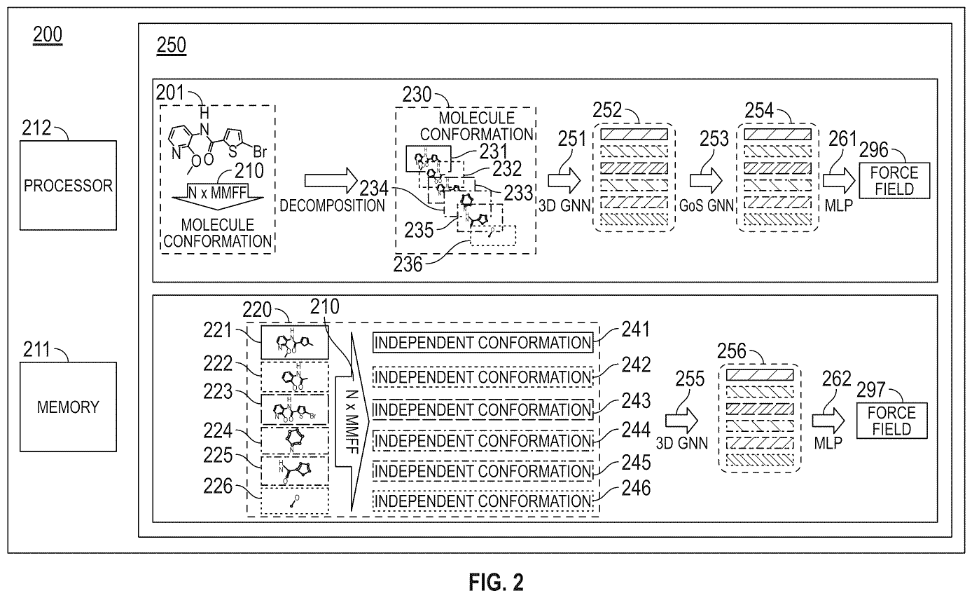

The material property prediction system 103 may also include a force field prediction system 125, which may implement the force field subgraph modeling functions as disclosed herein. For example, the force field prediction system 125 may pre-train the material subgraph model 120 to integrate force field subgraph modeling and/or force field predictions relating to the molecular structure of materials 106 into the material property predictions. Thus, the force field prediction system 125 may support the analysis and/or processing of force field data relating to the molecular structure of materials 106 that can potentially impact their chemical properties. The force field prediction system 125 may implement the force field subgraph modeling circuit 250 (see FIG. 2) and relate functions, as described in greater detail in reference to FIG. 2. In some embodiments, the force field subgraph modeling and/or force field predictions implemented by the force field prediction system 125 may be an auxiliary process with respect to material property prediction, such as pre-training the material subgraph model 120 in a manner where the force field computations are utilized downstream in subsequent material property subgraph modeling and/or prediction functions. In some embodiments, the force field subgraph modeling and/or force field predictions implemented by the force field prediction system 125 may be a stand-alone (e.g., asynchronous) process with respect to material property prediction functions, and may be performed by the force field prediction system 125 separately in addition to and/or in lieu of the material property prediction.

The material subgraph model 120 may be used to implement computational modeling techniques, which can be used to generate material property predictions based on the physical, chemical, electrical, mechanical, and/or optical properties of materials (e.g., the molecular structure of potential material). Subsequently, based on the enhanced material property predictions implemented by the material property prediction system 103, the material subgraph model 120, and/or the force field prediction system 125, the product design system 102 may be able to perform a plurality of functions (e.g., select, design, fabricate, validate, and/or the like) involving one or more of the raw materials 106 that may be deemed suitable and/or optimal to design (e.g., computer aided design) the product, which may ultimately be used for the manufacture of the product in the production line 108.

The product design system 102 may have the capability to perform aspects related to automated simulation, design, fabrication and/or validation of the one or more different organic materials 106 for each layer of the OLED display device. As an operational example, the product design system 102 may utilize the material subgraph model 120 in a computational prediction to determine and/or analyze key characteristics for the materials 106 such as light emission efficiency, stability, and charge transport properties prior to subsequent synthesis, testing, and/or utilization (e.g., for manufacturing products) of the materials 106. For example, the product design system 102 may utilize enhanced material property predictions (e.g., generated by the material property prediction system 103, material subgraph model 120, and/or force field prediction system 125) to select (e.g., molecules that may meet determined criteria for materials 106) and/or filter (e.g., molecules that may not meet determined criteria for materials 106) the materials 106 and/or molecular structure of materials 106 for subsequent designing, fabrication, validation, and/or utilization of materials 106.

In another example, the product design system 102 may control and/or execute functions involved in synthesizing the materials 106. For instance, the product design system 102 may control one or more automated functions for a chemical reactor to synthesize materials 106 using the molecules selected based on the enhanced material property predictions (e.g., generated by the material property prediction system 103, material subgraph model 120, and/or force field prediction system 125). The product design system 102 may control and/or execute a plurality of functions involved in material fabrication and/or synthesis for materials 106. Thus, enhanced material property prediction may be leveraged in fabrication and/or synthesis of materials 106 to increase the speed, efficiency, and performance of the process (e.g., improving the selected candidate molecules and/or materials, etc.) which may reduce the overall time consumed (e.g., delay between molecular design to validation of materials 106) and may optimize the design and/or performance of synthesized materials 106.

In another operational example, the product design system 102 may control and/or execute functions involved in automated testing and/or validation of the materials 106 based on the enhanced material property predictions (e.g., generated by the material property prediction system 103, material subgraph model 120, and/or force field prediction system 125)). For instance, the product design system 102 may control an automated test of synthesized materials 106 (e.g., OLED test materials), which may involve analyzing the emitted light from products using the materials 106 to measure the operational properties (e.g., electroluminescence) thereby validating and/or confirming (e.g., comparing measured properties of testing against theoretical and/or predetermined properties) the performance of the materials 106. Thus, enhanced material property prediction may be leveraged in validation and/or testing of materials 106 to ensure that the fabricated and/or synthesized materials 106 (and products manufactured using the materials 106) meet desired quality and/or criteria (e.g., color, luminance, and/or the like) prior to being utilized downline in the system 100, for instance in the production line 108 for manufacturing of display related products (e.g., synthesized materials are processed into films for layers).

In another operational example, the product design system 102 may control and/or execute functions downline in the system 100, for instance in the production line 108, involved in manufacture of the display related devices after testing and/or validating materials 106. For example, the product design system 102 may communicate (e.g., transmit) a command (e.g., control signal) to a system in the production line 108, such as a system of manufacturing and/or fabrication machines, to control executing an automated production of the OLED display device using the selected material (e.g., obtaining the selected material from raw materials 106) based on the enhanced material property predictions (e.g., generated by the material property prediction system 103, material subgraph model 120, and/or force field prediction system 125), thus optimizing the overall display performance and minimizing development time and cost.

FIG. 2 is a block diagram depicting a computer device 200 for force field prediction, including a force field subgraph modeling circuit 250 implementing force field subgraph modeling, according to some embodiments of the present disclosure.

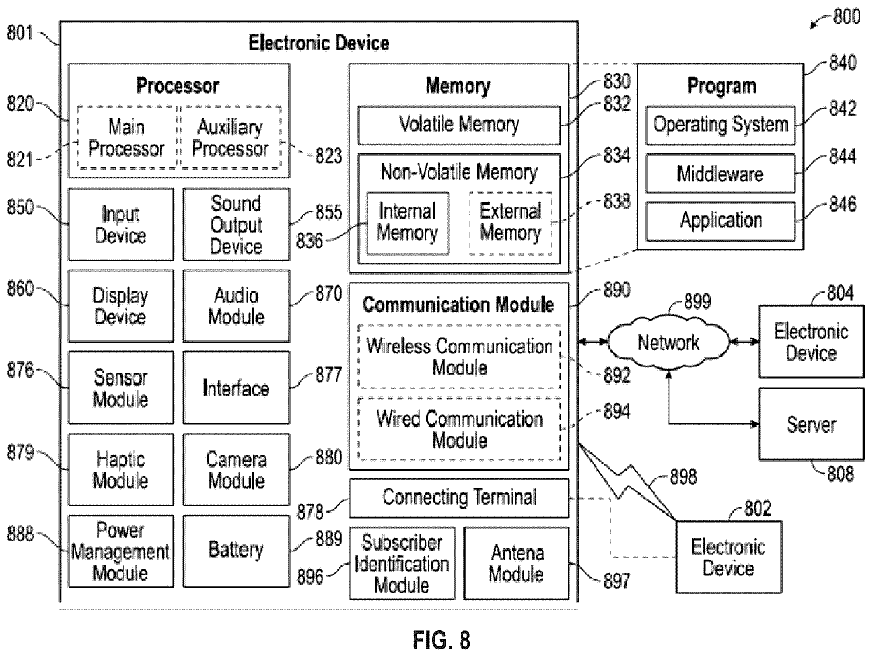

As illustrated in FIG. 2, the computer device 200 (e.g., one or more computers and/or one or more computer systems) may include a memory 211 (e.g., a memory and/or a storage), a processor 212, and a force field subgraph modeling circuit 250 configured for implementing subgraph modeling, as disclosed herein. The memory 211 may correspond to the memory 830 of FIG. 8. The processor 212 may correspond to the processor 820 of FIG. 8. As a general description, the force field subgraph modeling circuit 250 may execute one or more functions related to force field subgraph modeling, as disclosed herein, which may involve implementing decomposition of a molecular structure into subgraphs, performing force field calculations, obtaining embeddings of subgraphs, generating graphs of subgraphs, modeling interactions between subgraphs, and generating independent force field predictions and/or integrated force field predictions based on the subgraphs.

According to some embodiments, the computer device 200 may utilize AI-based models (e.g., material subgraph model 120) that are generated, pre-trained, trained, and/or utilized by the force field subgraph modeling circuit 250 and related functions, thus experiencing improved accuracy, efficiency, and/or performance. For example, the force field subgraph modeling circuit 250 may be configured to perform one or more of the related functions, as disclosed herein, during a training phase (including a force field pre-training) and/or during an inference phase of the AI-based models (e.g., material subgraph model 120).

The computer device 200 may include a computer system that is capable of AI-related functions including model training, computations, inference, and various AI-based applications. For example, the computer device 200 may be implemented as, for example, and without limitation, a desktop PC, a laptop, a smartphone, a tablet PC, a server, and/or the like. The computer device 200 may also refer to a system in which a cloud computing environment is established. However, some embodiments are not limited thereto. The computer device 200 may be implemented as any system, device, or apparatus which is capable of AI-based applications and functions (e.g., material property predictions, force field predictions (e.g., integrated and/or independent), subgraph modeling, etc.), such as the material property prediction system 103 and/or the force field prediction system 125 (e.g., shown in FIG. 1), as described herein. In some embodiments, the computer device 200 may implement various functions (e.g., material selection, product design, etc.) related to manufacturing and/or inspection of an OLED display device, such as a production design system 102 (e.g., shown in FIG. 1), as disclosed herein. The computer device 200 may include one or more processors for performing one or more of the processes of the present disclosure.

In some embodiments, the memory 211 may store data and/or AI models associated with AI-based applications, as disclosed herein (e.g., material property prediction, force field predictions (e.g., integrated and/or independent), subgraph modeling, etc.), including material subgraph model 120. In some embodiments, the memory 211 may store models generated, pre-trained, trained, and/or utilized by the force field subgraph modeling circuit 250. In some embodiments, the memory 211 may store the force field predictions (e.g., integrated and/or independent) generated by the force field subgraph modeling circuit 250, and utilizing force field subgraph modeling functions, as disclosed herein.

In some embodiments, the processor 212 may include various processing circuitry and may control overall operations of the computer device 200, including AI-based applications supported by the AI models (e.g., material subgraph model 120) generated, pre-trained, trained, and utilized by the force field subgraph modeling circuit 250, as disclosed herein. In some embodiments, the processor 212 may include various processing circuitry (e.g., one or more processing circuits) and may control overall operations of the computer device 200 (e.g., the computer system), including AI-based applications supported by the integrated force field predictions 296 and/or the independent force field predictions 297 generated by the force field subgraph modeling circuit 250, as disclosed herein. In some embodiments, the processor 212 may be implemented, for example, and without limitation, as a digital signal processor (DSP), a microprocessor, or a time controller (TCON), or the like, but is not limited thereto. The processor 212 may, for example, and without limitation, be one or more of a dedicated processor, a central processing unit (CPU), a micro controller unit (MCU), a micro processing unit (MPU), a controller, an application processor (AP), a communication processor (CP), an ARM processor, or the like, or may be defined as one of the terms above. Also, the processor 212 may be implemented as a system on chip (SoC) in which a processing algorithm is provided, or may be implemented in a form of a field programmable gate array (FPGA), or the like, but is not limited thereto.

The force field subgraph modeling circuit 250 may be implemented utilizing any suitable hardware, firmware (e.g. an application-specific integrated circuit), software, or a combination of software, firmware, and hardware. The material subgraph modeling circuit 250 may be configured to implement force field calculations, as described in greater detail herein. As used herein, “force field calculations” may refer to models, mathematical algorithms, and/or computational functions that may be utilized to analyze molecular conformations, energies, and interactions, including force fields. The force field subgraph modeling circuit 250 may be configured to implement various force field related calculations that may be pertinent to molecular structure of materials, including, but not limited to: force field minimization, energy, bond stretching, angle bending, torsional strain, electrostatic interactions, Van der Wals forces, and/or the like. In some embodiments, the force field subgraph modeling circuit 250 is configured to execute the force field calculations utilizing Merk Molecular Force Field (MMFF) calculations. Embodiments described herein are not limited thereto, and the force field subgraph modeling circuit 250 may be configured to implement force field calculations related to the molecular structure of a material utilizing any functions known in the art as deemed suitable and/or optimal.

The force field subgraph modeling circuit 250 may be configured to perform decomposition to generate multiple subgraphs, including molecular substructures of the material, from a graph of a molecular structure (e.g., graph of entire molecule) of the material. For example, the force field subgraph modeling circuit 250 may be configured to utilize chemical decomposition techniques, such as BRIC, hierarchical decomposition strategies, and/or the like, to extract molecular substructures related to materials. Thus, the force field material subgraph modeling circuit 250 may be configured to perform the extraction of functional groups, substructures, and/or structural motifs that may be critical to determining force fields, molecular conformations, and/or material properties, and generate subgraphs including these extracted (or decomposed) substructures.

The force field subgraph modeling circuit 250 may be configured to utilize decomposition functions and force field calculation functions, as previously described, in order to implement integrated force field predictions and/or independent force field predictions. As illustrated in FIG. 2, the force field subgraph modeling circuit 250 may perform force field calculations (e.g., MMFF function 210) that are applied to the input 201, which may be a graph of the molecular structure (e.g., prior to decomposition) to generate an integrated molecule conformation 230, which can be utilized to generate the integrated force field prediction 296. The force field subgraph modeling circuit 250 may perform force field calculations (e.g., MMFF function 210) that that are applied to independent subgraphs 220, which may include molecular substructures (e.g., after decomposition) to generate independent substructure conformations 241-246, which can be utilized to generate the independent force field prediction(s) 297.

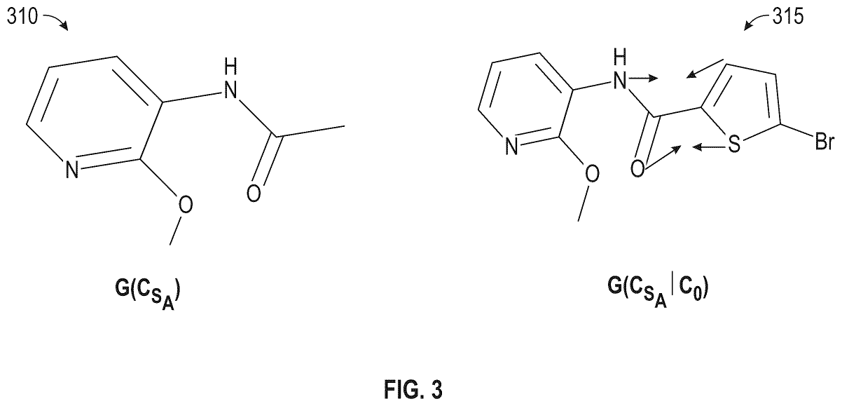

FIG. 3 depicts a conceptual diagram of an independent force field 310 related to a molecular structure of a material and an integrated force field 315 related to a molecular structure of a material. Independent force field 310 illustrates an example of minimization of an independent force field 310 that may be achieved when a molecular substructure (e.g., a subgraph A) is isolated from other substructures (e.g., subgraph A independent from other subgraph sections), and its force field may be minimized without considering any interactions or constraints imposed by other parts of the molecule. The resulting substructure conformation may reflect the intrinsic stability and a preferred geometry of the substructure, which may be based solely on its own chemical bonds and interactions.

Independent force field 315 illustrates an example of minimization of an integrated force field 315 that may be achieved when the molecular substructure (e.g., subgraph A) remains a part of the entire molecular graph, and its conformation may be influenced by interactions with other substructures in the molecule (e.g., subgraph A included with other subgraph sections within the entire graph). The presence of additional bonds (from other substructure in the molecule), steric hindrance, and electronic interactions can alter the preferred geometry of the substructure (compared to its isolated state).

The force field subgraph modeling circuit 250 may be configured to implement graphing of subgraphs which provide a modular (e.g., having multiple segments and/or portions) and 3D graph-based representation of molecules (e.g., molecular substructures), force fields, and properties, as described in greater detail herein.

The force field subgraph modeling circuit 250 may be configured to implement modeling of interactions between subgraphs, as described in greater detail herein. Thus, the force field subgraph modeling circuit 250 may capture the dependencies and/or interactions among subgraphs by leveraging the graph-based representation of subgraphs, preserving relational information and can achieve improved accuracy in force field prediction by maintaining the relational interactions. By modeling interactions between subgraphs, the force field subgraph modeling circuit 250 may provide an enhanced expressiveness level for AI models (e.g., greater than that of the 3-Weisfeiler-Lehman (3-WL) test), which can enable the AI models to distinguish complex and large-scale graph structures in a manner that may improve upon the capabilities of simpler models.

FIG. 2 illustrates that the force field subgraph modeling circuit 250 may be configured to receive an input 201, which may include data representing a molecular structure of material (e.g., chemicals) related to the production and/or manufacturing of a product. In some embodiments, the input 201 may be an initial graph representation of the molecular structure of the material (e.g., graph of entire molecule), where the chemical composition of the material can include large and complex molecules (e.g., having hundreds of atoms). The material subgraph modeling circuit 250 may utilize the input 201 to perform integrated force field functions that may ultimately generate the integrated force field prediction 296 and/or or independent force field functions that may ultimately generate the independent force field prediction(s) 297. For example, with respect to integrated force field functions, the force field subgraph modeling circuit 250 may apply (e.g., iteratively) the MMFF function 210, for example (as the force field calculation functon), to the input 201 which may be the graph of the molecule (prior to decomposition), and then may subsequently perform decomposition to generate an integrated molecule conformation 230 (including subgraphs 231-236). Also, with respect to independent force field functions, the force field subgraph modeling circuit 250 may perform decomposition to the input 201 to generate independent subgraphs 221-226 of molecular substructures (e.g., each of the subgraphs 221-226 may include a different substructure from the entire graph of the molecule), and then may apply (e.g., iteratively) the MMFF function 210 to each of the subgraphs 221-226 to generate an independent substructure conformations 241-246. It should be appreciated that the operations performed with respect to integrated force field functions and/or independent force field functions implemented by the force field subgraph modeling circuit 250 may be performed serially, in parallel, cooperatively (in addition), and/or independently (in lieu of) each other, in entirety and/or in combination, in accordance with some of the embodiments.

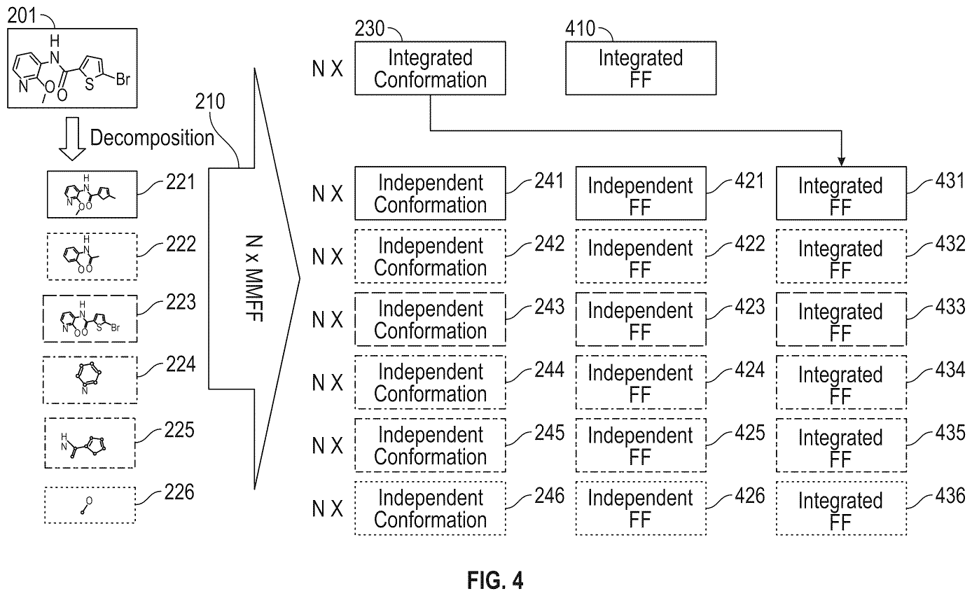

FIG. 4 is a conceptual diagram depicting an example process 400 for implementing decomposition and force field calculations that may be implemented by the force field subgraph modeling circuit 250, according to some embodiments.

FIG. 4 illustrates that the process 400 may be implemented by the force field subgraph modeling circuit 250 to generate: the integrated molecule conformation 230 and integrated force field calculation(s) 410; and the independent substructure conformations 241-246 with corresponding independent force field calculation(s) 421-426 and integrated force field calculation(s) 431-436. In some embodiments, the independent force field calculation(s) 421-426 may be ultimately implemented as the independent force field prediction(s) 297 based on force field subgraph modeling functions described herein, the integrated force field calculation(s) 410 and/or the integrated force field calculation(s) 431-436 may be ultimately implemented as the integrated force field prediction 296 based on force field subgraph modeling functions described herein.

For example, with respect to integrated force field functions, the MMFF function 210 may be applied to the input 201, which may be a graph of the molecule, for a determined number (e.g., N) of iterations. In some implementations, the MMF function 210 is applied for at least 10 iterations (e.g., N≥10). However, embodiments are not limited thereto, and the number of iterations performed for the MMFF function 210 may be dynamically determined and/or set (e.g., pre-determined) as deemed suitable and/or optimal for optimized the structures, and/or deriving force field related calculations (e.g., computations, values, predictions, etc).

The result of applying (e.g., iteratively) the MMFF function 210 to the input 201 may be the generation of the integration conformation 230 and the integrated force field calculation(s) 410. The integrated conformation 230 may represent the spatial arrangement of atoms in the structure of the entire molecular, incorporating the positioning and/or arrangement of multiple substructures therewithin. Accordingly, force field calculations that are based on analysis of the molecular graph (e.g., as a whole structure) may be performed, where the integrated force field calculation(s) 410 can capture interactions between substructures within the molecule (e.g., presence of additional bonds, steric hindrance, and electronic interactions, etc.).

Also, with respect to independent force field functions, the process 400 may involve decomposing the input 201 (e.g., graph of molecule) into multiple substructures, where each substructure may be represented as one of the independent subgraphs 231-236.

For example, the force field subgraph modeling circuit 250 may execute decomposition of the molecular structure represented in the input 201 to extract one or more molecular substructures, where the molecular substructures may further be represented as subgraphs 231-236. In some embodiments, the force field modeling circuit 250 may be configured to implement BRIC for decomposing the molecule represented by the input 201. The force field subgraph modeling circuit 250 may perform decomposition, in accordance with BRIC, to identify molecular substructures, atoms, bonds, and/or other conformation related elements within molecules of the material that may be critical to the chemical structure and/or disclosed functions (e.g., material property prediction, force filed prediction, etc.). Decomposition may be based on a determined number (e.g., minimum, maximum, etc.) of functional groups and/or structural motifs to be extracted. The independent subgraphs 231-236 may then be generated based on the substructures resulting from decomposition.

In some embodiments, the force field subgraph modeling circuit 250 may be configured to control and/or determine the number of subgraphs 231-236 that are generated from molecular substructures as a result of the decomposition. For example, the force field subgraph modeling circuit 250 may generate a determined number of subgraphs 231-236 such that the aggregated composition of the subgraphs 231-236 may cover the original graph of the molecule represented by the input 201. The number of subgraphs 231-236 generated from decomposition may be determined based on application related factors, including but not limited to: computational resources and/or configuration of the system 200; efficiency and/or performance related metrics (e.g., maintain the computational speed); and/or the like. In some embodiments, the force field subgraph modeling circuit 250 may be configured to prioritize one or more molecular substructures to be selected for extracting during decomposition. For instance, a specific substructure of a molecule may be deemed as chemically significant to the molecular structure of a material, and thus may be prioritized by the force field subgraph modeling circuit 250 over other molecular substructures with respect to being extracting during decomposition and represented in subgraphs 231-236. The embodiments are not limited thereto, and the force field subgraph modeling circuit 250 may be configured to utilize other material and/or molecular decomposition mechanisms as deemed suitable and/or appropriate.

The decomposition may involve systematically fragmenting the molecule in the input 201 by identifying parts, regions (or subregions), and/or breaking bonds as deemed suitable (e.g., breaking bonds of retrosynthetic significance, etc.). For example, decomposition may involve decomposing the molecule based on identifying elements related to the confirmation such as, rings, non-cyclic parts, and carbon-carbon single bonds, and/or the like, that may comprise the molecule. The molecular substructures formed from decomposing the complex structure of molecule represented in the input 201 may be represented as subgraphs 231-236. In the example of FIG. 4, each of the independent subgraphs 231-236 may be generated to include (or represent) a different substructure of the molecule that resulted from the decomposition.

The MMFF function 210 may be applied (e.g., iteratively and/or independently) to each of the independent subgraphs 231-232 (including a different substructure), which may generate independent substructure conformations 241-246. FIG. 4 illustrates that each of the subgraphs 231-236 have one of the substructure conformations 241-246, one of the independent force field calculations 421-426, and one of the integrated force field calculations 431-436 corresponding thereto. The substructure conformations 241-246 may represent the spatial arrangement of atoms in the respective molecular substructure, isolating the positioning and/or arrangement of substructure (from other of the multiple substructures within the entire structure of the molecule). Accordingly, force field calculations that are based on analysis of the substructures in subgraphs may be performed, where the independent force field calculations 421-426 may be based on an isolated and/or independent substructure (e.g., without considering any interactions or constraints imposed by other parts of the molecule), and the integrated force field calculations 431-436 can capture interactions between substructures within the molecule (e.g., as a whole structure from the perspective of the respective substructure). In some embodiments, the integrated force field calculation(s) 410 may be utilized in the computations and/or analysis to derive the integrated force field calculations 431-436.

Referring again to FIG. 2, the force field subgraph modeling circuit 250 may be configured to obtain subgraph embeddings 252 by processing the integrated molecule conformation 230 (based on subgraphs 231-236) utilizing a graph neural network (GNN) (e.g., three-dimensional (3D) GNN 251), and may obtain subgraph embeddings 256 by processing the independent substructure conformations 241-246 (based on subgraphs 221-226) utilizing a GNN (e.g., 3D GNN 255). For example, the force field subgraph modeling circuit 250 may input one or more of the subgraphs 220 (representing molecular substructure) and/or one or more of the independent substructure conformations 241-246 into the 3D GNN 255 to be analyzed and subsequently generate the subgraph embeddings 256, where each of the subgraph embeddings 256 can be a vector representation of a portion of the molecule's (input 201) properties from the corresponding subgraph 220 (including the respective substructure).

Furthermore, the force field subgraph modeling circuit 250 may be configured to obtain updated subgraph embeddings 254 by processing the subgraph embeddings 252 utilizing a graph of subgraphs GNN 253 as disclosed herein.

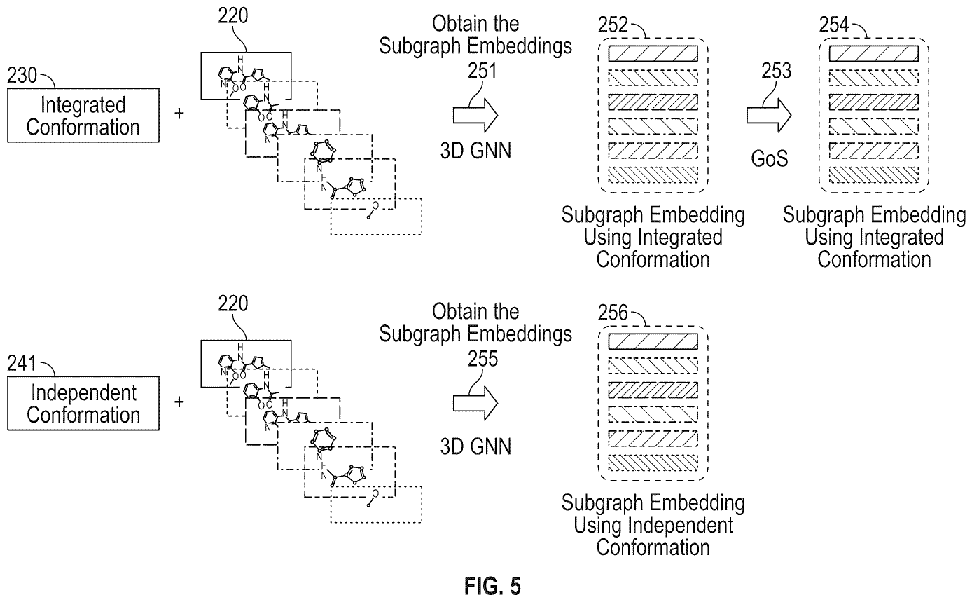

FIG. 5 depicts an example of a process for obtaining subgraph embeddings 252, 256 by utilizing 3D GNNs 251, 255 and obtaining subgraph embeddings 254 by utilizing a graph of subgraphs 253 implemented by the fore field subgraph modeling circuit 250, according to some embodiments of the present disclosure.

The 3D GNNs 251, 255 may implement an 3D AI-based processing of graph-structured data (e.g., nodes, edges, etc.) such as the subgraphs including data representing molecular structures and/or substructures, and 3D related information (e.g., 3D spatial coordinates (x, y, z), Euclidean distance, angular relationships, etc.) that may be captured by molecular conformations. As used herein, a “3D GNN” may refer to a type of graph neural network that may be designed to process and/or analyze data that has 3D spatial structure (e.g., connections, distance, positions in the 3D space) and/or relationships, and can be utilized for implementing molecular modeling (e.g., force field modeling, etc.), as disclosed herein.

As an example, each of the substructure subgraphs 220 may be structured as a set of nodes and edges. Additionally, the integrated conformation 230 and an independent substructure conformation 241 may represent 3D relation information (e.g., spatial relationships, 3D coordinates, etc.) for the molecule and its substructures. The 3D GNNs 251, 255 may be configured to receive the 3D conformation of the molecule and substructures, together with the graph related properties (e.g., node property, edge property, etc.) as input. FIG. 5 illustrates an example with the 3D GNN 251 receiving the integrated conformation 230 and the subgraphs 220 as input; and the 3D GNN 255 receiving the independent substructure conformation 241 and the subgraphs 220 as input.

The 3D GNN 251, 255 may receive the information and analyze the subgraphs 220 and conformations 230, 241 in order to capture features, attributes, 3D positions and/or 3D spatial relationship, encodings, and/or the like that relate to the data (e.g., nodes), relationships therebetween (e.g., edges), molecular arrangements (e.g., conformations) within the learned subgraph embeddings 252 (using integrated confirmation) and subgraph embeddings 256 (using independent conformations). The subgraph embeddings 252, 256 may be obtained in a format that a machine learning model can understand and utilize for downstream tasks. Functions performed by the 3D GNNs 251, 255 to obtain the subgraph embeddings 252, 256 may involve tokenization, embedding, incorporating spatial information, positional encoding, and/or the like. In some embodiments, the 3D GNN 251, 255 may receive subgraphs 220 and conformations 230, 241-246 and obtain subgraph embeddings 252, 256 iteratively as a function of a training process (e.g., pre-training) and/or during inference for a machine learning model, being executed by the processor 112.

In some embodiments, the force field subgraph modeling circuit 250 may be configured to generate a graph of subgraphs 253 as another representation of the subgraphs 220. In some embodiments, the graph of subgraphs 253 neural network that can analyze and/or generate the molecular data as a graphical structure (representing data as a set of nodes and edges, where each of the nodes within the constructed graph of subgraphs 253 may represent a corresponding subgraph 220 and the edges may represent interactions and/or relationships between the corresponding subgraphs 220). In some embodiments, the graph of subgraphs 253 may analyze and/or generate the molecular data (from the subgraph embeddings 252) in a manner where a node may represent a subgraph 220, the value of a node may correspond to one of the subgraph embeddings 252, and the edges may represent interactions and/or relationships between the corresponding subgraphs 220). In some embodiments, a structure of the graph of subgraphs 253 may be represented as an adjacency matrix, where each cell of the adjacency matrix may indicate a structural attribute of the subgraph 220 (e.g., whether an edge exists between two nodes, etc.).

In some embodiments, the graph of subgraphs 253 may be a neural network that may calculate and/or analyze the relationships and/or interactions (e.g., including 3D spatial relationship and/or interactions) between the subgraphs 220 in a manner that can model the relationship through hierarchical relations. Distance methods, such as Jaccard distance, fingerprints, and/or the like may be used to calculate the relationships between subgraphs 220, and the relational dependencies may be graphically represented in the structure of the graph of subgraphs 253 (e.g., number of connections and/edges, distance between nodes, etc.). For instance, the edge may represent that there is a relational dependency between the node (representing a corresponding subgraph 220) and the node (representing another corresponding subgraph 220). In some embodiments, the neural network implementing the graph of subgraphs 253 may be an Equivariant Graph Neural Network (EGNN) that is configured to process and/or analyze 3D coordinates and/or related information. Accordingly, processing the graph of subgraphs 253 through the neural network may model the relationships and/or interactions (in the 3D space) between molecular substructures (included in the subgraphs 220) that may be pertinent to the force field. Thus, the neural network implementing the graph of subgraphs 253 may be utilized for capturing and modeling the interactions and/or dependencies between the subgraphs 220 (and substructures) in a manner that can preserve relational information and enable the representation of complex structural patterns, such as the complex molecular structure of materials for products.

In some embodiments, the material subgraph modeling circuit 250 may be configured to implement updated subgraph embeddings 254 by utilizing the graph neural network implementing the graph of subgraphs 253.

In some embodiments, the GNN implementing the graph of subgraphs 253 may utilize multiple layers to iteratively update the received subgraph embeddings 252, for example by aggregating information from neighboring nodes within the graph of subgraphs 253. A passing of information from a subgraph (e.g., a node) to neighboring subgraphs (e.g., a neighboring node) may be performed for updating. After a determined number of iterations (layers), the GNN implementing the graph of subgraphs 253 may obtain the updated embeddings 254 (based on the integrated conformation 230). In some embodiments, the updated embeddings 254 may be also aggregated by the GNN implementing the graph of subgraphs 253, which can be generated a single vector (representing the aggregation of the updated embeddings 254).

Referring again to FIG. 2, the force field modeling circuit 250 may be configured to pass the updated subgraph embeddings 254 (based on the integrated conformation 230), and the subgraph embeddings 256 to a respective multi-layer perception (MLP) function 261, 262. The MLP functions 261, 262 may be a transformation function that processes embeddings for further processing tasks, such as classification, regression, and/or the like, and outputs the respective integrated force field prediction(s) 296, and the independent force field prediction(s) 297. As previously described, the force field subgraph modeling circuit 250 may achieve a force fielding modeling that effectively models long-range interactions associated with molecular structure through the relational modeling between subgraphs in a manner that leverages diversity (e.g., integrated fore field functions, independent force field functions, multiple conformations, etc.) to improve the model's robustness and increase the accuracy and/or performance of the force field predictions 296, 297.

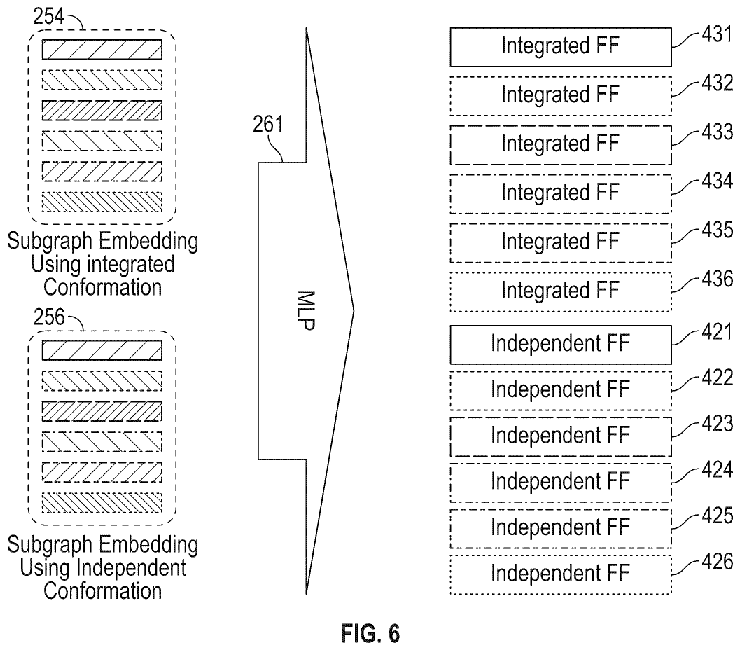

FIG. 6 depicts an example of a process for generating integrated force field predictions 431-436 and independent force field predictions 421-421 from embeddings 254, 256 that may be implemented by the force field modeling circuit 250, according to some embodiments of the present disclosure.

As previously described, the updated subgraph embeddings 254 (based on the integrated molecule conformation 230) and the subgraph embeddings 256 (based on the independent conformations 241-246) may be generated as spatially (3D space) aware embeddings that may capture the relationships between subgraphs, thereby modeling the relationships between the molecular substructures. In some embodiments, the computational results (e.g., calculations of the last layer of GNNs) may be captured by the subgraph embeddings 254, 256, and these subgraph embeddings 254, 256 may be passed to the MLP function 261, which processes the result from the embeddings 254, 256 for further processing tasks, such as classification, regression, and/or the like, and outputs the respective integrated force field predictions 431-436 based on the integrated force field operations (utilizing integrated molecule conformation 230) executed by the force field subgraph circuit 250, and outputs the independent force field predictions 421-426 based on the independent force field operations (utilizing independent substructure conformations 241-246) executed by the force field modeling circuit.

In some embodiments, a loss associated with the force field predictions 421-426, 431-436 generated by the AI-based inference and/or prediction functions executed by the force field modeling circuit 250 may may be represented as a mean square error (MSE) between the predicted force field, and true force field. In some embodiments, the subgraph embeddings 254, 256, and the force field predictions 421-426, 431-436 may be utilized by downstream operations and/or functions, including but not limited to: pre-training of the material subgraph model 120; generating AI-based material property predictions; material selection for production of a display related product; and/or the like. In some embodiments, output force field predictions 421-426, 431-436 may be a result and/or prediction of an auxiliary and/or stand-alone AI-based process.

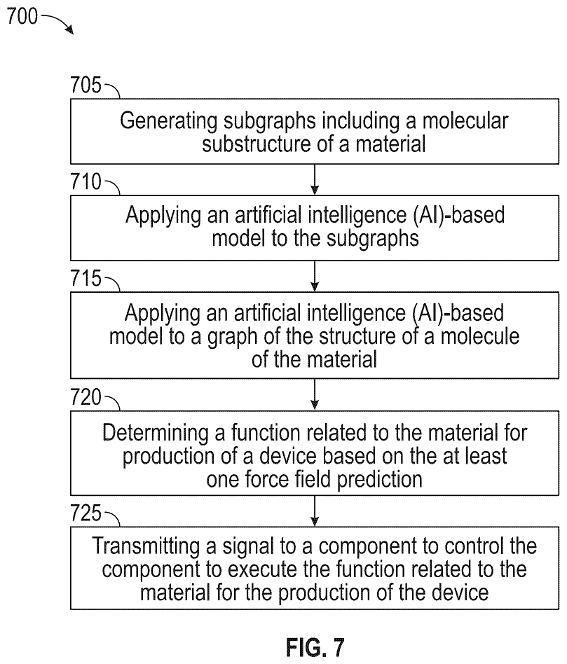

FIG. 7 is a flow chart depicting example operations of a method 700 for utilizing AI-based force field subgraph models and/or enhanced force field predictions, according to some embodiments of the present disclosure. For example, FIG. 7 illustrates various operations in a method 700 for force field subgraph models and/or enhanced force field predictions, according to some embodiments. Although FIG. 7 illustrates various operations in a method according to some embodiments, embodiments according to the present disclosure are not limited thereto, and according to various embodiments, the method may include additional operations or fewer operations without departing from the spirit and scope of embodiments according to the present disclosure.

According to some embodiments, and as discussed in more detail above, the method 700 may include, at operation 705, generating subgraphs including a molecular substructure of a material. At operation 710, the method 700 may further include applying an artificial intelligence (AI)-based model to the subgraphs to generate at least one force field prediction based on the molecular substructure of the material. At operation 715, the method 700 may further include applying the AI-based model to a graph of a structure of a molecule of the material to generate a force field prediction based on the structure of the molecule of the material. At operation 720, the method 700 may further include determining a function related to the material for production of a device based on the at least one prediction based on the molecular substructure and the force field prediction based on the structure of a molecule of the material. The function may be related to design, fabrication and/or synthesis, testing and/or validation, and/or utilization (e.g., during manufacturing of an OLED device) of the material.

After determining to the function related to the material for production of a device, the method 700 may further include, at operation 720, transmitting a signal to a component (e.g., in a production line) to control the component to execute the function related to the material for the production of the device. For example, according to some embodiments, transmitting the signal may include transmitting a control signal to a manufacturing component operating as part of a production line that may physically retrieve, handle, and/or process the material (e.g., in an automated fashion, without manual human intervention) as part of a manufacturing process of a device. Additionally, according to some embodiments, transmitting the signal may include transmitting a signal to computer system including a display device operating as a manufacturing component as part of a production line or production facility to display a result of the determination thereon.

FIG. 8 is a block diagram of an electronic device in a network environment, according to some embodiments of the present disclosure.

Referring to FIG. 8, an electronic device 801 in a network environment 800 may communicate with an electronic device 802 via a first network 898 (e.g., a short-range wireless communication network), or with an electronic device 804 or a server 808 via a second network 899 (e.g., a long-range wireless communication network). The electronic device 801 may communicate with the electronic device 804 via the server 808. The electronic device 801 may include a processor 820, a memory 830, an input device 850, a sound output device 855, a display device 860, an audio module 870, a sensor module 876, an interface 877, a haptic module 879, a camera module 880, a power management module 888, a battery 889, a communication module 890, a subscriber identification module (SIM) card 896, and/or an antenna module 897. In one embodiment, at least one of the components (e.g., the display device 860 or the camera module 880) may be omitted from the electronic device 801, or one or more other components may be added to the electronic device 801. Some of the components may be implemented as a single integrated circuit (IC). For example, the sensor module 876 (e.g., a fingerprint sensor, an iris sensor, or an illuminance sensor) may be embedded in the display device 860 (e.g., a display).

The processor 820 may execute software (e.g., a program 840) to control at least one other component (e.g., a hardware or a software component) of the electronic device 801 coupled to the processor 820, and may perform various data processing or computations.

As at least part of the data processing or computations, the processor 820 may load a command or data received from another component (e.g., the sensor module 876 or the communication module 890) in volatile memory 832, may process the command or the data stored in the volatile memory 832, and may store resulting data in non-volatile memory 834. The processor 820 may include a main processor 821 (e.g., a central processing unit or an application processor (AP)), and an auxiliary processor 823 (e.g., a graphics processing unit (GPU), an image signal processor (ISP), a sensor hub processor, or a communication processor (CP)) that is operable independently from, or in conjunction with, the main processor 821. Additionally, or alternatively, the auxiliary processor 823 may be adapted to consume less power than the main processor 821, or to execute a particular function. The auxiliary processor 823 may be implemented as being separate from, or a part of, the main processor 821.

The auxiliary processor 823 may control at least some of the functions or states related to at least one component (e.g., the display device 860, the sensor module 876, or the communication module 890), as opposed to the main processor 821 while the main processor 821 is in an inactive (e.g., sleep) state, or together with the main processor 821 while the main processor 1821 is in an active state (e.g., executing an application). The auxiliary processor 823 (e.g., an image signal processor or a communication processor) may be implemented as part of another component (e.g., the camera module 880 or the communication module 890) functionally related to the auxiliary processor 823.

The memory 830 may store various data used by at least one component (e.g., the processor 820 or the sensor module 876) of the electronic device 801. The various data may include, for example, software (e.g., the program 840) and input data or output data for a command related thereto. The memory 830 may include the volatile memory 832 or the non-volatile memory 834.

The program 840 may be stored in the memory 830 as software, and may include, for example, an operating system (OS) 842, middleware 844, or an application 846.

The input device 850 may receive a command or data to be used by another component (e.g., the processor 820) of the electronic device 801, from the outside (e.g., a user) of the electronic device 801. The input device 850 may include, for example, a microphone, a mouse, or a keyboard.

The sound output device 855 may output sound signals to the outside of the electronic device 801. The sound output device 855 may include, for example, a speaker or a receiver. The speaker may be used for general purposes, such as playing multimedia or recording, and the receiver may be used for receiving an incoming call. The receiver may be implemented as separate from, or as a part of, the speaker.

The display device 860 may visually provide information to the outside (e.g., to a user) of the electronic device 801. The display device 860 may include, for example, a display, a hologram device, or a projector, and may include control circuitry to control a corresponding one of the display, hologram device, and projector. The display device 860 may include touch circuitry adapted to detect a touch, or may include sensor circuitry (e.g., a pressure sensor) adapted to measure the intensity of force incurred by the touch.

The audio module 870 may convert a sound into an electrical signal and vice versa. The audio module 870 may obtain the sound via the input device 850 or may output the sound via the sound output device 1855 or a headphone of an external electronic device 802 directly (e.g., wired) or wirelessly coupled to the electronic device 801.

The sensor module 876 may detect an operational state (e.g., power or temperature) of the electronic device 801, or an environmental state (e.g., a state of a user) external to the electronic device 801. The sensor module 876 may then generate an electrical signal or data value corresponding to the detected state. The sensor module 876 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, and/or an illuminance sensor.

The interface 877 may support one or more specified protocols to be used for the electronic device 801 to be coupled to the external electronic device 802 directly (e.g., wired) or wirelessly. The interface 877 may include, for example, a high-definition multimedia interface (HDMI), a universal serial bus (USB) interface, a secure digital (SD) card interface, or an audio interface.

A connecting terminal 878 may include a connector via which the electronic device 801 may be physically connected to the external electronic device 802. The connecting terminal 878 may include, for example, an HDMI connector, a USB connector, an SD card connector, or an audio connector (e.g., a headphone connector).

The haptic module 879 may convert an electrical signal into a mechanical stimulus (e.g., a vibration or a movement) or an electrical stimulus, which may be recognized by a user via tactile sensation or kinesthetic sensation. The haptic module 879 may include, for example, a motor, a piezoelectric element, or an electrical stimulator.

The camera module 880 may capture a still image or moving images. The camera module 880 may include one or more lenses, image sensors, image signal processors, or flashes. The power management module 888 may manage power that is supplied to the electronic device 801. The power management module 888 may be implemented as at least part of, for example, a power management integrated circuit (PMIC).

The battery 889 may supply power to at least one component of the electronic device 801. The battery 889 may include, for example, a primary cell that is not rechargeable, a secondary cell that is rechargeable, or a fuel cell.

The communication module 890 may support establishing a direct (e.g., wired) communication channel or a wireless communication channel between the electronic device 801 and the external electronic device (e.g., the electronic device 802, the electronic device 804, or the server 808), and may support performing communication via the established communication channel. The communication module 890 may include one or more communication processors that are operable independently from the processor 820 (e.g., the AP), and may support a direct (e.g., wired) communication or a wireless communication. The communication module 890 may include a wireless communication module 892 (e.g., a cellular communication module, a short-range wireless communication module, or a global navigation satellite system (GNSS) communication module) or a wired communication module 894 (e.g., a local area network (LAN) communication module or a power line communication (PLC) module). A corresponding one of these communication modules may communicate with the external electronic device via the first network 898 (e.g., a short-range communication network, such as BLUETOOTH®, wireless-fidelity (Wi-Fi) direct, or a standard of the Infrared Data Association (IrDA)), or via the second network 899 (e.g., a long-range communication network, such as a cellular network, the Internet, or a computer network (e.g., LAN or wide area network (WAN)). These various types of communication modules may be implemented as a single component (e.g., a single IC), or may be implemented as multiple components (e.g., multiple ICs) that are separate from each other. The wireless communication module 892 may identify and authenticate the electronic device 801 in a communication network, such as the first network 898 or the second network 899, using subscriber information (e.g., international mobile subscriber identity (IMSI)) stored in the subscriber identification module 896.

The antenna module 897 may transmit or receive a signal or power to or from the outside (e.g., the external electronic device) of the electronic device 801. The antenna module 897 may include one or more antennas. The communication module 890 (e.g., the wireless communication module 1892) may select at least one of the one or more antennas appropriate for a communication scheme used in the communication network, such as the first network 1898 or the second network 899. The signal or the power may then be transmitted or received between the communication module 890 and the external electronic device via the selected at least one antenna.

Commands or data may be transmitted or received between the electronic device 801 and the external electronic device 804 via the server 808 coupled to the second network 899. Each of the electronic devices 802 and 804 may be a device of a same type as, or a different type, from the electronic device 801. All or some of operations to be executed at the electronic device 801 may be executed at one or more of the external electronic devices 802, 804, or 808. For example, if the electronic device 801 should perform a function or a service automatically, or in response to a request from a user or another device, the electronic device 801, instead of, or in addition to, executing the function or the service, may request the one or more external electronic devices to perform at least part of the function or the service. The one or more external electronic devices receiving the request may perform the at least part of the function or the service requested, or an additional function or an additional service related to the request and transfer an outcome of the performing to the electronic device 801. The electronic device 801 may provide the outcome, with or without further processing of the outcome, as at least part of a reply to the request. To that end, cloud computing, distributed computing, or client-server computing technology may be used, for example.

Accordingly, aspects of some embodiments of the present disclosure may provide systems and/or functions related to AI-based force field subgraph models in a manner that may incorporate accuracy and/or robust force field information (e.g., predictions) relating to molecules of a material to further improve the accuracy of downstream tasks (e.g., material property predictions) related to the production of a display related product. Thus, the disclosed embodiments may improve the overall performance of display related products by considering the impacts of force fields and/or utilizing materials deemed most suitable and/or efficient.

Embodiments of the subject matter and the operations described in this specification may be implemented in digital electronic circuitry, or in computer software, firmware, or hardware, including the structures disclosed in this specification and their structural equivalents, or in combinations of one or more of them. Embodiments of the subject matter described in this specification may be implemented as one or more computer programs, i.e., one or more modules of computer-program instructions, encoded on computer-storage medium for execution by, or to control the operation of data-processing apparatus. Alternatively or additionally, the program instructions can be encoded on an artificially-generated propagated signal, e.g., a machine-generated electrical, optical, or electromagnetic signal, which is generated to encode information for transmission to suitable receiver apparatus for execution by a data processing apparatus. A computer-storage medium can be, or be included in, a computer-readable storage device, a computer-readable storage substrate, a random or serial-access memory array or device, or a combination thereof. Moreover, while a computer-storage medium is not a propagated signal, a computer-storage medium may be a source or destination of computer-program instructions encoded in an artificially-generated propagated signal. The computer-storage medium can also be, or be included in, one or more separate physical components or media (e.g., multiple CDs, disks, or other storage devices). Additionally, the operations described in this specification may be implemented as operations performed by a data-processing apparatus on data stored on one or more computer-readable storage devices or received from other sources.

While this specification may contain many specific implementation details, the implementation details should not be construed as limitations on the scope of any claimed subject matter, but rather be construed as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described herein. Other embodiments are within the scope of the following claims. In some cases, the actions set forth in the claims may be performed in a different order and still achieve desirable results. Additionally, the processes depicted in the accompanying figures do not necessarily require the particular order shown, or sequential order, to achieve desirable results. In certain implementations, multitasking and parallel processing may be advantageous.

As will be recognized by those skilled in the art, the innovative concepts described herein may be modified and varied over a wide range of applications. Accordingly, the scope of claimed subject matter should not be limited to any of the specific exemplary teachings discussed above, but is instead defined by the following claims.

What is claimed is:

1. A method comprising:
generating, by a processor, subgraphs, each of the subgraphs comprising a molecular substructure of a material;
applying, by the processor, an artificial intelligence (AI)-based model to the subgraphs to generate at least one force field prediction based on the molecular substructure of the material;
applying, by the processor, the AI-based model to a graph of a structure of a molecule of the material to generate a force field prediction based on the structure of the molecule of the material;
determining, by the processor, a function related to the material for production of a device based on the at least one force field prediction based on the molecular substructure of the material and the force field prediction based on the structure of the molecule of the material; and
transmitting, by the processor, a signal to a component to control the component to execute the function related to the material for the production of the device.

2. The method of claim 1, further comprising applying, by the processor, the AI-based model to the subgraphs to generate a material property prediction, wherein generating the at least one force field prediction based on the molecular substructure of the material and generating the force field prediction based on the structure of the molecule of the material is a pre-training process of the AI-based model.

3. The method of claim 1, further comprising decomposing the graph of the structure of the molecule into the molecular substructures.

4. The method of claim 2, wherein the decomposing comprises Breaking Retrosynthetically Interesting Chemical bonds (BRIC) decomposition.

5. The method of claim 3, further comprising applying a force field calculation function to the subgraphs, and obtaining at least one substructure conformation.

6. The method of claim 5, further comprising generating subgraph embeddings by processing the subgraphs and the at least one substructure conformation by a three-dimensional (3D) graph neural network.

7. The method of claim 6, wherein the at least one force field prediction based on the molecular substructure of the material is generated based on the subgraph embeddings.

8. The method of claim 1, further comprising applying a force field calculation function to the graph of the structure of the molecule, and obtaining a molecule conformation.

9. The method of claim 8, further comprising generating subgraph embeddings by processing the subgraphs and the molecule conformation by a three-dimensional (3D) graph neural network.

10. The method of claim 9, further comprising generating a graph of subgraphs based on the subgraph embeddings.

11. The method of claim 10, wherein the graph of subgraphs comprises nodes and edges.

12. The method of claim 11, wherein each of the nodes correspond to one of the subgraphs and a value for each of the nodes correspond to one of the embeddings of the subgraphs.

13. The method of claim 12, wherein each of the edges represent a relationship between the subgraphs.

14. The method of claim 13, further comprising generating updated subgraph embeddings based on the graph of subgraphs processing the graph of subgraphs by a graph neural network.

15. The method of claim 14, wherein the force field prediction based on the structure of the molecule of the material is generated based on the updated subgraph embeddings.

16. The method of claim 11, wherein the AI-based model analyzes the graph of subgraphs and models relationships between the subgraphs.

17. The method of claim 11, wherein the device comprises an organic light-emitting diode (OLED) display device.

18. A device comprising:
one or more processors that are configured to perform:
generating subgraphs, each of the subgraphs comprising a molecular substructure of a material;
applying an artificial intelligence (AI)-based model to the subgraphs to generate at least one force field prediction based on the molecular substructure of the material;
applying the AI-based model to a graph of a structure of a molecule to generate a force field prediction based on the structure of the molecule of the material;
determining a function related to the material for production of a device based on the at least one force field prediction based on the molecular substructure of the material and the force field prediction based on the structure of the molecule of the material; and
transmitting a signal to a component to control the component to execute the function related to the material for the production of the device.

19. The device of claim 18, wherein the one or more processors are further configured to apply the AI-based model to the subgraphs to generate a material property prediction, wherein generating the at least one force field prediction based on the molecular substructure of the material and generating the force field prediction based on the structure of the molecule of the material is a pre-training process of the AI-based model.

20. A system comprising:
a processing circuit; and
a non-volatile memory storing instructions, which, based on being executed by the processing circuit, cause the processing circuit to perform:
generating subgraphs, each of the subgraphs comprising a molecular substructure of a material;
applying an artificial intelligence (AI)-based model to the subgraphs to generate at least one force field prediction based on the molecular substructure of the material;
applying the AI-based model to a graph of a structure of a molecule of the material to generate a force field prediction based on the structure of the molecule of the material;
determining a function related to the material for production of a device based on the at least one force field prediction based on the molecular substructure of the material and the force field prediction based on the structure of the molecule of the material; and
transmitting a signal to a component to control the component to execute the function related to the material for the production of the device.
